(19) United States
(12) Patent Application Publication
(10) Pub. No.: US 2026/0290773 A1
(43) Pub. Date: Sep. 24, 2026

(54) METHODS FOR DETERMINING KINETIC ENERGY LOSS IN CHARGE DETECTION MASS SPECTROMETRY

(71) Applicant: THERMO FINNIGAN LLC, San Jose, CA (US)

(72) Inventors: Michael Goodwin, Santa Clara, CA (US); Michael Senko, Sunnyvale, CA (US)

(21) Appl. No.: 19/085,243

(22) Filed: Mar. 20, 2025

(51) Int. Cl. H01J 49/02; G01N 33/68; H01J 49/42

(52) U.S. Cl. CPC H01J 49/027; G01N 33/6848; H01J 49/425

(57) ABSTRACT

The present disclosure generally provides methods for determining a kinetic energy loss. The methods include injecting an ion population including the ion of interest into a trapping region and establishing an electrostatic trapping field within the region. A time-varying signal representative of a current induced on a detector based on the ion population is generated. A Selective Temporal Overview of Resonant Ion (STORI) plot is generated based on the time-varying signal. An ion decay event is identified, in which the ion decay event includes a kinetic energy loss occurring in the trapping region.

### BACKGROUND OF THE INVENTION

### Field of the Invention

The invention relates generally to mass spectrometry, and more particularly to apparatus and methods for measurement of the mass-to-charge ratio and charge of an ion.

### Description of Related Art

Charge detection mass spectrometry (CDMS) is a technique where the masses of individual ions are determined from concurrent measurement of each ion's mass-to-charge ratio (m/z) and charge. Conventional techniques for performing CDMS include the use of Fourier Transform (FT) mass spectrometry (FT-MS). In FT-MS, ions oscillate in an electrostatic field (e.g., in an orbital electrostatic trap mass analyzer, such as an Orbitrap® analyzer), in a magnetic field (e.g., in an FT-Ion Cyclotron Resonance (FT-ICR) mass analyzer), or a combination of both. A signal measured in time, referred to as a transient, is Fourier transformed to obtain a frequency domain spectrum. An ion's oscillation frequency is a function of the ion's mass and, hence, m/z. Thus, each spectral component (peak) in the frequency domain spectrum corresponds to a particular oscillation frequency of a trapped ion, from which the ion's m/z may be unambiguously determined. An ion's charge z may then be determined directly from the amplitude of the frequency domain peak based on the assumption that the induced current signal is proportional to the ion's charge state. A simple CDMS approach would assume that the amplitude of the frequency domain peak for a given frequency would be proportional to the ion's charge.

However, the frequency domain peak amplitude is not a good measure of the induced current signal because the ion's dwell time (lifetime) in the analyzing trap may be shorter than the signal acquisition period. An ion's lifetime may be shortened by a destructive collision with a molecule of a residual gas or by other mechanisms of ion fragmentation. As a result of fragmentation, the ion is either removed from the set of trapped ions or its oscillation frequency changes abruptly due to a change in mass. In both cases, the induced-current signal terminates at the original frequency. In the case that a charged fragment with a different m/z remains trapped in the analyzer, a new frequency domain component appears in the induced current signal at a different frequency at the same moment when the original signal from the original ion is lost. The new frequency, although different than the original frequency, may be indistinguishable in the frequency domain spectrum from the original frequency. However, as the new signal component starts not at the beginning of the acquisition period, its peak height is also underestimated.

Previous attempts to estimate an ion's actual lifetime within the signal acquisition period have been implemented, e.g., the traditional Selective Temporal Overview of Resonant Ion (STORI) or the misSTORI method. The methods give accurate estimates of the rate at which an ion's signal is increasing with time, as opposed to the overall signal that is induced. This is helpful when ions die partway through a transient, such that simply using their overall absolute signal could be ambiguous. The STORI and misSTORI techniques have mostly been utilized in the detection of ions within orbital electrostatic trap mass analyzer. However, each of these methods do not account for varying image current induced by an ion due to the dependency of the ion's trajectory within the analyzer. For example, an ion that flies closer to the detection electrodes will induce more current than an ion that flies further away from the electrodes, even if the two ions possess the same charge. The exact trajectory of an ion inside an orbital electrostatic trap mass analyzer depends on numerous factors, including m/z, initial kinetic energy and trajectory when entering the orbital electrostatic trap mass analyzer, collisional kinetic energy losses when entering the orbital electrostatic trap mass analyzer, kinetic energy and trajectory when in the orbital electrostatic trap mass analyzer, and collisional kinetic energy losses when in the orbital electrostatic trap mass analyzer.

Indeed, the ion will progressively lose kinetic energy and will induce lower signal as time progresses as the transient acquisition increase due to the likelihood for more collisions. If the collision frequency is high enough and the collisional losses are sufficiently strong, this effect can be significant, making ions appear to have less charge and therefore less mass. It can be especially problematic if using very long transients, where the number of collisions (and therefore magnitude of kinetic energy losses) grows.

Accordingly, what is needed is an improved method for determining kinetic energy loss in charge detection mass spectrometry.

### SUMMARY OF THE INVENTION

The present disclosure generally provides methods for determining a kinetic energy loss. The methods include injecting an ion population including the ion of interest into a trapping region and establishing an electrostatic trapping field within the region. A time-varying signal representative of a current induced on a detector based on the ion population is generated. A Selective Temporal Overview of Resonant Ion (STORI) plot is generated based on the time-varying signal. An ion decay event is identified, in which the ion decay event includes a kinetic energy loss occurring in the trapping region.

The present disclosure also generally provides methods for determining a kinetic energy loss. The methods include storing an ion population including an ion of interest in an ion store. The ion population is injected into a trapping region and an electrostatic trapping field is established within the region. A time-varying signal representative of a current induced on a detector based on the ion population is generated. A Selective Temporal Overview of Resonant Ion (STORI) plot is generated based on the time-varying signal. An ion decay event is identified, in which the ion decay event includes a kinetic energy loss.

The present disclosure also generally provides methods for determining a collision cross section of an ion of interest. The methods include injecting an ion population including the ion of interest into a trapping region and establishing an electrostatic trapping field within the region. A time-varying signal representative of a current induced on a detector based on the ion population is generated. A Selective Temporal Overview of Resonant Ion (STORI) plot is generated based on the time-varying signal. A collision cross section is determined of the ion of interest.

### BRIEF DESCRIPTION OF THE DRAWINGS

The above noted and various other aspects of the present invention will become apparent from the following description which is given by way of example only and with reference to the accompanying drawings, not necessarily drawn to scale.

FIG. 1 is a symbolic diagram of an apparatus for concurrent measurement of the m/z and charge of an ion, according to aspects of the present disclosure.

FIG. 2 is a block diagram depicting logical components of the data system of FIG. 1, according to aspects of the present disclosure.

FIG. 3 is a graphical representation of a STORI plot for a single ion.

FIG. 4 is a graphical representation of a STORI plot for a single ion that decays during the signal acquisition period.

FIG. 5 is a graphical representation of a mass decline over time with varying trapping gas settings, according to aspects of the present disclosure.

FIG. 6 is a graphical representation of a mass decline rate over collision cell pressure, according to aspects of the present disclosure.

FIG. 7 is a graphical representation of signal over kinetic energy to charge, according to aspects of the present disclosure.

FIG. 8 is a graphical representation of a kinetic energy rate of change over a collision cell pressure, according to aspects of the present disclosure.

FIG. 9 is a graphical representation of a theoretical kinetic energy rate of change compared to an experimental kinetic energy rate of change, according to aspects of the present disclosure.

### DETAILED DESCRIPTION

The present disclosure generally provides methods for determining kinetic energy loss in charge detection mass spectrometry (CDMS). The present disclosure can identify pressure influences on the absolute mass peak position by extrapolating a “zero time”, e.g., a time at which the kinetic energy losses have not yet occurred, thereby accounting for kinetic energy losses in CDMS. The method can include calibrating and/or adjusting the absolute mass peak position based on the collisional kinetic energy losses of an ion in charge detection mass spectrometry, e.g., collisional kinetic energy losses of an ion in an orbital electrostatic trap mass analyzer system. Moreover, the present disclosure can be implemented according to a plurality of collisional cross sections of analytes, thereby allowing for a plurality of calibration corrections to be implemented based on the collision cross section of each respective ion and/or analyte. Additionally, the present disclosure can determine accurate charge assignments due to the calibration, thereby further clarifying the mass assignment of the ion in an orbital electrostatic trap mass analyzer system.

Advantageously, the present disclosure can improve charge precision in CDMS processes and can determine a collision cross section for an ion and/or analyte during CDMS processes.

The following description is presented to enable any person skilled in the art to make and use the invention, and is provided in the context of a particular application and its requirements. Various modifications to the described embodiments will be readily apparent to those skilled in the art and the generic principles herein may be applied to other embodiments. Thus, the present invention is not intended to be limited to the embodiments and examples shown but is to be accorded the widest possible scope in accordance with the features and principles shown and described.

In the description of the invention herein, it is understood that a word appearing in the singular encompasses its plural counterpart, and a word appearing in the plural encompasses its singular counterpart, unless implicitly or explicitly understood or stated otherwise. Furthermore, it is understood that, for any given component or embodiment described herein, any of the possible candidates or alternatives listed for that component may generally be used individually or in combination with one another, unless implicitly or explicitly understood or stated otherwise. Moreover, it is to be appreciated that the figures, as shown herein, are not necessarily drawn to scale, wherein some of the elements may be drawn merely for clarity of the invention. Also, reference numerals may be repeated among the various figures to show corresponding or analogous elements. Additionally, it will be understood that any list of candidates or alternatives is merely illustrative, not limiting, unless implicitly or explicitly understood or stated otherwise.

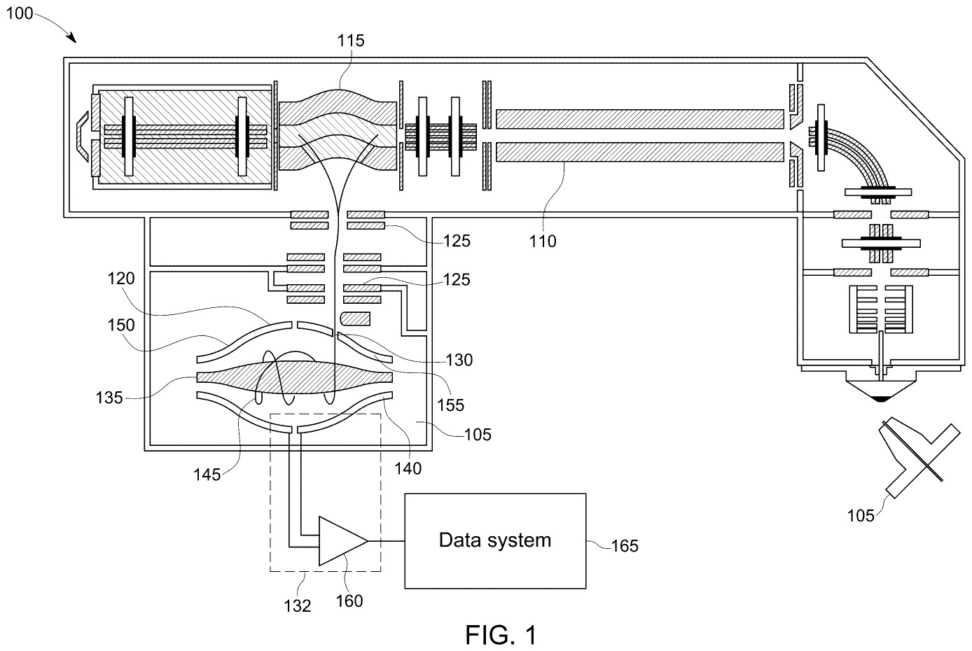

FIG. 1 symbolically depicts a mass spectrometry apparatus 100 arranged in accordance with one embodiment of the present invention. Apparatus 100 includes an ionization source 105 that generates ions from a sample to be analyzed. As used herein, the term “ion(s)” refers to any charged molecule or assembly of molecules, and is specifically intended to embrace high molecular weight entities sometimes referred to in the art as macro-ions, charged particles, and charged aerosols. Without limiting the scope of the invention, ions that may be analyzed by apparatus 100 including a protein, a protein complex, an antibody, a viral capsid, an oligonucleotide, a high molecular weight polymer, and/or a combination thereof. Source 105 may take the form of an electrospray ionization (ESI) source, in which the ions are formed by spraying charged droplets of sample solution from a capillary to which a potential is applied. The sample may be delivered to source 105 as a continuous stream, e.g., as the eluate from a chromatographic column.

Ions generated by source 105 are directed and focused through a series of ion optics disposed in vacuum chambers of progressively reduced pressures. As depicted in FIG. 1, the ion optics may include ion transfer tubes, stacked ring ion guides, radio-frequency (RF) multipoles, and electrostatic lenses. The vacuum chambers in which the ion optics are contained may be evacuated by any suitable pump or combination of pumps operable to maintain the pressure at desired values.

Apparatus 100 may additionally include a quadrupole mass filter (QMF) 110 that transmits only those ions within a selected range of values of m/z. The operation of quadrupole mass filters is well known in the art and need not be discussed in detail herein. Generally described, the m/z range of the selectively transmitted ions is set by appropriate adjustment of the amplitudes of the RF and resolving direct current (DC) voltages applied to the electrodes of QMF 110 to establish an electric field that causes ions having m/z's outside of the selected range to develop unstable trajectories. The transmitted ions may thereafter traverse additional ion optics (e.g., lenses and RF multipoles) and enter ion store 115. As is known in the art, ion store 115 employs a combination of oscillatory and static fields to confine the ions to its interior. In a specific implementation, ion store 115 may take the form of a curved trap (referred to colloquially as a “c-trap”) of the type utilized in Orbitrap mass spectrometers sold by Thermo Fisher Scientific. The curved trap is composed of a set of generally parallel rod electrodes that are curved concavely toward the ion exit. Radial confinement of ions within ion store 115 may be achieved by applying oscillatory voltages in a prescribed phase relationship to opposed pairs of the rod electrodes, while axial confinement may be effected by applying static voltages to end lenses positioned axially outwardly of the rod electrodes.

Ions entering ion store 115 may be confined therein for a prescribed cooling period in order to reduce their kinetic energies prior to introduction of the ions into electrostatic trap. Confinement of the ions within the ion store for a prescribed period may also assist in desolvation of the ions, i.e., removal of any residual solvent moieties from the analyte ion. As discussed hereinabove, the presence of residual solvent may result in mass shifts during analysis which interfere with the ability to accurately measure m/z and charge. To facilitate kinetic cooling and desolvation of the ions, an inert gas such as argon or helium may be added to the ion store internal volume; however, the cooling gas pressure should be regulated to avoid unintended fragmentation of the analyte ions and/or excessive leakage of the gas into electrostatic trap 120. The duration of the cooling period will depend on a number of factors, including the kinetic energy of ions entering ion store 115, the inert gas pressure, and the desired kinetic energy profile of ions injected into electrostatic trap 120. After the cooling period has been completed, ions confined in ion store 115 may be radially ejected from ion store toward entrance lenses 125, which act to focus and direct ions into inlet aperture 130 of electrostatic trap 120. Rapid ejection of ions from ion store 115 may be performed by rapidly collapsing the oscillatory field within the ion store interior and applying a DC pulse to the rod electrodes positioned away from the direction of ejection.

To reliably measure ion charge using the CDMS technique, only individual ions of a particular ion species can be present in electrostatic trap 120 during a measurement event. As used herein, the term “ion species” refers to an ion of a given elemental/isotopic composition and charge state; ions of different elemental/isotopic compositions are considered to be different ion species, as well as are ions of the same elemental composition but different charge states. The term “ion species” is used interchangeably herein with the terms “analyte ion(s)” and “ion(s) of interest”. If multiple ions of the same ion species are present during a measurement event, then the measured charge state (determined from the amplitude of the signal generated by image current detector 132, as described below) will be a multiple of the actual charge state of an individual ion. To avoid this type of mismeasurement, the ion population within ion store 115 should be kept sufficiently small such that the likelihood that two ions of the same ion species are confined within the ion store is maintained at an acceptable minimum. This may be accomplished by attenuation of the ion beam generated by source 105 (more specifically, by “detuning” ion optics located in the upstream ion path such that high losses of ions occur) and/or via regulation of the fill time (the period during which ions are accepted into ion store 115), as described in McGee, John P., et al. “Automated Control of Injection Times for Unattended Acquisition of Multiplexed Individual Ion Mass Spectra.” Analytical chemistry 94.48 (2022): 16543-16548, the entirety of which is incorporated herein. To control the fill time, one or more ion optic components located upstream in the ion path of ion store may be operated as a gate to selectively allow or block passage of ions into the internal volume of ion store 115.

Electrostatic trap 120 may take the form of an orbital electrostatic trap, of the type commercially available from Thermo Fisher Scientific under the trademark “Orbitrap” and depicted in cross-section in FIG. 1. Such orbital electrostatic traps include an inner electrode 135 defining a central longitudinal axis, designated in a cylindrical coordinate system as the z-axis. An outer electrode 140 is positioned coaxially with respect to inner electrode 135, defining therebetween a generally annular trapping region 145 into which ions are injected. The outer electrode 140 surrounds the inner electrode 135. Inner electrode 135 and outer electrode 140 are each symmetrical about a transverse plane (designated as z=0, and alternatively referred to as the “equator”), with inner electrode 135 having a maximum outer radius of R₁ and outer electrode 140 having a maximum inner radius of R₂ at the transverse plane of symmetry. As has been discussed widely in the scientific literature (see, e.g., Makarov, “Electrostatic Axially Harmonic Orbital Trapping: A High-Performance Technique of Mass Analysis”, Analytical Chemistry, Vol. 72, No. 6, pp. 1156-62 (2000), which is incorporated herein by reference), the inner and outer electrodes may be shaped to establish (upon application of electrostatic voltage(s) to one or both of the electrodes) an electrostatic potential distribution U(r,z), within trapping region 145 that approximates the relation, where U(r,z) is defined by:

$$U ( r , z ) = \frac{k}{2} ( {z}^{2} - \frac{{r}^{2}}{2} ) + \frac{k}{2} * ( {R}_{m} ) * ln ( \frac{r}{{R}_{m}} ) + C$$

where is the position of the ion of interest along a radial axis, z is the position of the ion of interest along a central axis (r=0 being the central longitudinal axis and z=0 being the transverse plane of symmetry), C is a constant, k is field curvature, and Rₘ is the characteristic field radius. This field is sometimes referred to as a quadro-logarithmic field.

Outer electrode 140 is split along the transverse plane of symmetry into first and second parts 150 and 155, which are separated from each other by a narrow insulating gap. This arrangement enables the use of outer electrode 140, together with differential amplifier 160, as an image current detector. The presence of an ion proximal to the outer electrode induces a charge (of a polarity opposite to that of the ion) in the electrode having a magnitude proportional to the charge of the ion. The oscillatory back-and-forth movement of an ion along the z-axis between the first 150 and second 155 parts of outer electrode 140 causes image current detector 132 to output a time varying signal (referred to as a “transient”) having a frequency equal to the frequency of the ion's longitudinal oscillation and an amplitude representative of the ion's charge.

Ions may be introduced tangentially into trapping region 145 through inlet aperture 130 formed in outer electrode 140. Inlet aperture 130 is axially offset (along the z-axis) from the transverse plane of symmetry, such that, upon introduction into trapping region 145, the ions experience a restorative force in the direction of the plane of symmetry, causing the ions to initiate longitudinal oscillation along the z-axis while orbiting inner electrode 135, as illustrated in FIG. 1. A salient characteristic of the quadro-logarithmic field is that its potential distribution contains no cross-terms in r and z, and that the potential in the z-dimension is exclusively quadratic. Thus, ion motion along the z-axis may be described as a harmonic oscillator (because the force along the z-dimension exerted by the field on the ion is directly proportional to the displacement of the ion along the z-axis from the transverse plane of symmetry) and is completely independent of the orbital motion. In this manner, the frequency of ion oscillation ω along the z-axis is simply related to the ion's mass-to-charge ratio (m/z) according to the relation:

$$ω = \sqrt{\frac{k}{m / z}}$$

Measurement of charge state and m/z, and consequent calculation of the product mass, proceeds by the acquisition and processing of the transient. Transient acquisition by detector 132 is initiated promptly after injection of the analyte ion(s), and continued for a predetermined transient length. The transient length required for accurate measurement of m/z and charge state will vary according to the analyte, as well as the physical and operational parameters of electrostatic trap 120. In general, the transient will need to be of adequate duration to allow the signal to be reliably distinguished from noise. For a typical analyte ion, it is anticipated that a satisfactory signal-to-noise ratio may be achieved using a commercially-available orbital trapping mass analyzer at a transient length of 500 milliseconds. It will be understood that the maximum transient length will be limited by the duration for which the analyte ion is stably trapped within trapping region 145, which is in part a function of the trapping region pressure.

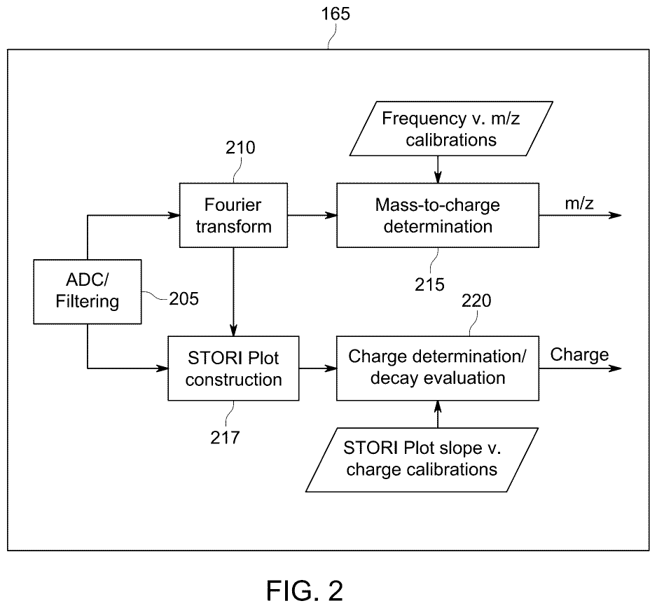

The transient signal produced by detector 132 is processed by data system 165, the functions of which will be described below in connection with FIG. 2. Although data system 165 is depicted as a unitary block, its functions may be distributed among several interconnected devices. Data system 165 will typically include a collection of specialized and general purpose processors, application specific circuitry, memory, storage, and input/output devices. Data system 165 is configured with logic, for example using executable software code, to perform a set of calculations to determine the fundamental frequency of the analyte ion's motion and to construct a STORI plot corresponding to the ion, which are used in turn to derive the m/z and charge state.

FIG. 2 depicts components of data system 165. Analog-to-digital converter (ADC) module 205 receives the analog signal generated by detector 132 and samples the signal at a prescribed sampling rate to generate a sequence of discrete time-intensity data values. ADC module 205 may also perform a filtering function to attenuate extraneous noise and improve signal-to-noise ratio. The time-domain data are then passed to a Fast Fourier transform (FFT) module 210 for conversion of the data into the frequency domain. FFT algorithms are well known in the art and hence need not be discussed in detail herein. Generally described, an FFT algorithm rapidly computes the discrete Fourier transform (DFT) of a sequence by factorizing the DFT matrix into a product of sparse factors. FFT module 210 generates as output a frequency spectrum, representing the decomposition of the time-domain data sequence into one or more frequency components, each frequency component comprising a single sinusoidal oscillation with its own amplitude.

The motion along the z-axis of an analyte ion trapped within the field generated in trapping region 145 is harmonic and may be represented as a simple sinusoidal function. The output of FFT module 210 will thereby yield a frequency spectrum that has a strong peak at the fundamental frequency of oscillation @ of the ion of interest. When multiple ion species are present within the electrostatic trap during the measurement event (e.g., during acquisition of a transient), then each ion species will exhibit a corresponding peak in the frequency spectrum. In contrast to prior art CDMS systems in which the oscillatory motion of a trapped ion is anharmonic and non-sinusoidal (for which the FFT output will include numerous peaks distributed among various harmonics), the signal for each ion species in the electrostatic trap 120 will be concentrated into a single peak appearing of the fundamental frequency of oscillation, thereby improving sensitivity and enabling charge measurement for lower-charge ions relative to prior art CDMS devices.

The frequency spectrum generated by the FFT module is provided as input to m/z determination module 215, which processes the frequency spectrum to determine the m/z of the analyte ion(s). M/z determination module 215 is configured to identify, for the or each analyte ion species present in the spectrum, the fundamental frequency of oscillation of the analyte ion. This frequency is then converted to a value of m/z. As noted above, the frequency of oscillatory ion motion along the z-axis is inversely proportional to the square root of the ion's m/z in accordance with the relation:

$$ω = \sqrt{\frac{k}{m / z}}$$

Thus, the m/z may be determined from the measured ion frequency using an empirically established frequency vs. m/z calibration curve generated by fitting an inverse square-root curve to data points acquired for analyte ions of known m/z, as is known in the art.

As described in further detail hereinbelow, charge determination module 220 is configured to process the STORI plot constructed by module 217 and provide as output, for the or each analyte ion species present in the spectrum, a value of the ion's charge.

Once the m/z and charge of the analyte ion has been determined, the mass of the ion may be calculated simply via the product of the determined m/z and charge. If the spectrum contains multiple ion species, the mass for each ion species is calculated by the product of the m/z and charge determined for that species.

In certain implementations, the transient acquisition and m/z and charge determination operations will be performed repeatedly for an analyte ion. The resultant calculated masses may be binned to obtain a mass histogram, with the peak of the histogram representing the most likely mass. Generally, the width of the histogram will depend on the accuracy of the image charge determination, with narrower widths being indicative of high accuracy. Other techniques, including averaging, may be employed to improve the reliability of mass determination.

### Use of STORI Plots for Charge Determination in CDMS

In CDMS, the ability to assign charge, and thus mass, accurately is dependent on the ability to determine the amplitude of the signal corresponding to the ion of interest. In the case where the ion generates signal throughout the signal acquisition period, the determination of the signal amplitude is accomplished simply via the amplitude of the resulting peak in the frequency domain.

However, ions may “decay” (disintegrate) during the acquisition period, resulting in the destabilization of the ion's trajectory. This can either be due to collision with a background gas molecule, or simply because the ion is metastable. If the ion decays during the acquisition period, less signal may be generated, with that signal being proportional to the lifetime of the ion, in which a reduction in signal may reduce the accurate mass determination. Therefore, in order to convert the frequency domain amplitude back to the undecayed time domain amplitude, one must be able to accurately correct for collisional kinetic energy losses both prior to being introduced to the orbital electrostatic trap mass analyzer and after being introduced to the orbital electrostatic trap mass analyzer.

A calculation (using the charge determination module described above, or such other data system component as may be suitable for the purpose) of a Selective Temporal Overview of Resonant Ion (STORI) plot is performed, alternatively referred to as Correlated Integral Profile (CIP) processing. For example, the time domain data is multiplied by a sine wave of the frequency of interest, and the output is the dot product of the two. Each point in a STORI plot is the product of the discretized time-varying signal S at time tₙ, and either a sine wave (equation 1, below) or cosine wave (equation 2, below) at the frequency of movement of the ion of interest ω (determinable from the Fourier transform of the time-domain signal data), summed with the prior STORI point obtained at prior time point tₙ₋₁, as expressed in the following equations.

$${STORI}_{REAL} ( {t}_{n} ) = S ( {t}_{n} ) * cos ( ω * {t}_{n} ) + {STORI}_{REAL} ( {t}_{n - 1} ) \qquad ( 1 )$$

$${STORI}_{IMAG} ( {t}_{n} ) = - S ( {t}_{n} ) * sin ( ω * {t}_{n} ) + {STORI}_{IMAG} ( {t}_{n - 1} ) \qquad ( 2 )$$

The foregoing components are each dependent on the initial phase of the signal, and thus neither component alone can provide quantitative information about the signal amplitude. The phase dependency of the signal can be removed by calculating the magnitude of the real and imaginary STORI components, as set forth below in equation (3):

$${STORI}_{MAG} ( {t}_{n} ) = {( {( {STORI}_{REAL} ( {t}_{n} ) )}^{2} + {( {STORI}_{IMAG} ( {t}_{n} ) )}^{2} )}^{1 / 2} \qquad ( 3 )$$

In the data system 165 depicted in FIG. 2, the foregoing calculations are performed by the operation of STORI plot construction module 217 (e.g., by execution of a set of software instructions), which receives the discretized time-domain signal data as input and outputs a representation of plot of STORI_{MAG} versus time. Where multiple analyte ions of different masses are present and it is desired to separately determine mass for each of the multiple analyte ions, then a STORI plot is constructed for each analyte ion, in accordance with their individual frequencies of movement (which vary in relation to their m/z's as described above). The STORI plot(s) may then be utilized by charge determination module 220 for determination of charge state, and for identification and characterization of ion decay events that occur during the acquisition of the time-varying signal. The STORI plot construction module 217 may also include logic for causing the calculated STORI plot to be visually displayed to the instrument operator on a monitor that constitutes part of data system 165.

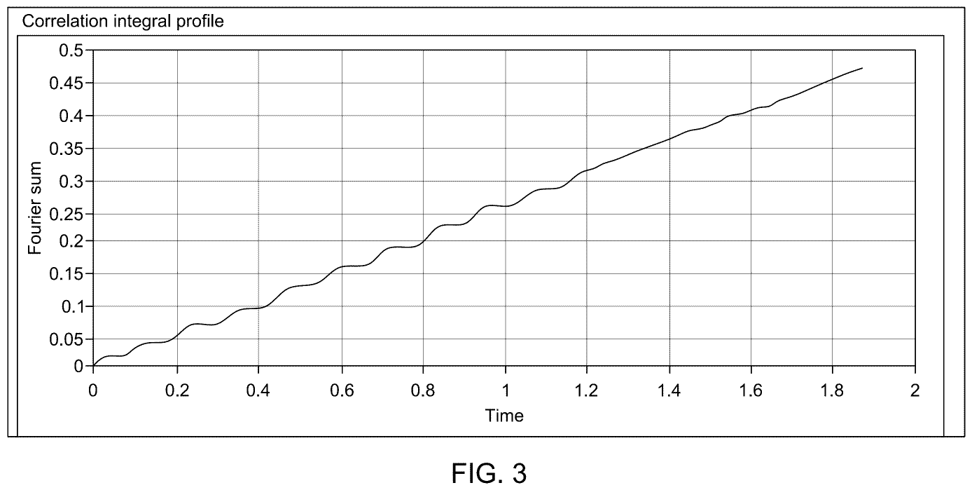

An example of a STORI plot for a single ion is depicted in FIG. 3, wherein the ion of interest generates signal over the entire signal acquisition period. In this STORI plot, the variation of STORI_{MAG} with time approximates a straight line with a constant slope. The STORI plot slope is a measure of ion charge, with ions of higher charge exhibiting a steeper slope relative to ions of lower charge. Thus, the charge state of an ion can be determined based on the slope of this line. In the FIG. 2 configuration, the charge state determination is performed by module 220 using a set of stored empirically derived calibrations relating STORI plot slope and charge state obtained using analytes of known charge. Since the STORI plot slope v. charge relationship may vary according to the operating conditions of the mass analyzer (for example, the voltage applied to inner electrode 135), the calibration data may be multi-dimensional, with slope v. charge relationships empirically established for different values of instrument operating parameters across an expected range.

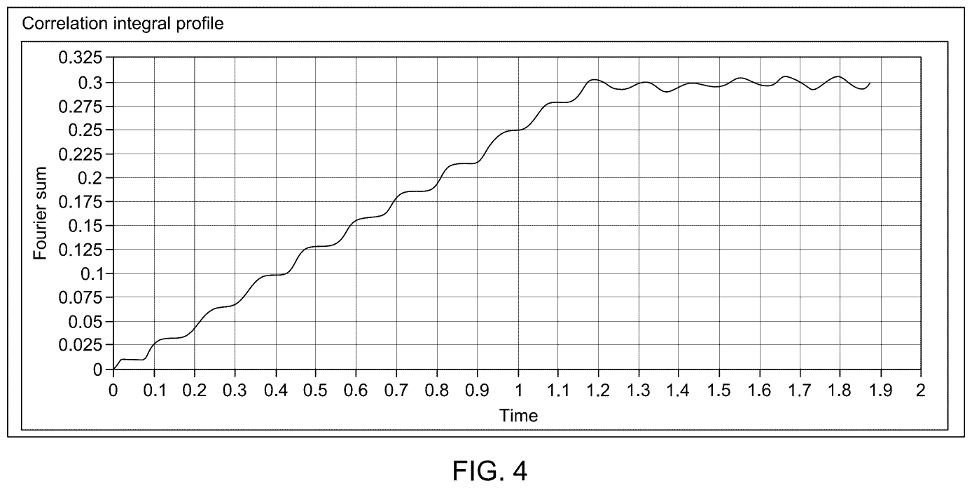

The shape of the STORI plot is also useful to reveal the occurrence of ion decay events. In FIG. 4, the STORI plot is shown for a single ion which happens to decay at approximately 1.2 seconds. In the standard Fourier transform, the peak that results from this decayed ion would have a reduced intensity relative to that derived from the Fourier transform of the ion from FIG. 3. This might lead one to believe that the ion of FIG. 4 has a charge that is lower than actuality. However, examination of the STORI plots of FIGS. 3 and 4 show that the slopes of the plots preceding a time point of about 1.2 seconds are the same, and thus both ions have the same charge state. In certain embodiments, STORI plot construction module 217 or charge determination module 220 may contain logic for evaluating the STORI plot and providing an indication to the operator that a decay event has occurred (i.e., responsive to detection of a change of slope across the acquisition period), or may contain logic for disregarding the post-decay portion of the STORI plot when determining charge. In other embodiments, where transient acquisition and m/z and charge determination steps are performed repeatedly for an ion of interest to generate a histogram of the distribution of measured masses, as described above, the STORI plot construction or charge determination module may discard (i.e., not include in the histogram construction) any transients where a change of slope in the STORI plot is observed over the acquisition period.

Visual inspection of the STORI plot depicted in FIG. 3 shows an initially “wiggly” (i.e., oscillating slightly about a straight line) portion, which oscillation substantially disappears after ~1.2 seconds. This wiggly behavior is actually due to the simultaneous presence in the trapping region of the electrostatic trap of the ion corresponding to the STORI plot of FIG. 4, which results in a repeating pattern of constructive and destructive interference. Depending on the point at which the slope is measured during the period of constructive and destructive interference, this can result in an improper estimate of charge state. This can be resolved by considering (i.e., via the operation of charge determination module 220) only the slope corresponding to the portion of the plot after the second ion has decayed, or by more sophisticated processing, where the slope of the STORI plot in FIG. 3 is measured across complete periods of the interference, which is described in U.S. patent application U.S. Ser. No. 18/643,938, entitled “Methods for performing charge detection mass spectrometry with temporal resolution”, the entirety of which is incorporated herein.

In an embodiment, an ion decay event may include a kinetic energy decay when entering an orbital electrostatic trap. In some embodiments, the energy of an ion may decay during one or more of trapping, cooling, and ejecting said ion from an ion store that is prior to the orbital electrostatic analyzer. The kinetic energy of the ion may be removed by collisions with neutral atoms or molecules (e.g. nitrogen) while it is being ejected from the ion store and/or in flight to the orbital electrostatic trap. The amount of kinetic energy lost will depend on numerous factors (e.g. the mass of the ion, the mass of the neutral, etc.). In some embodiments, the ion may enter the orbital electrostatic trap such that the ion orbits a center electrode of the orbital electrostatic trap at a closer distance than an ion that did not undergo as many collisions during transfer to the orbital electrostatic trap. An ion that orbits a center electrode closer may induce less image current at a detection electrode, thereby inducing a reduced charge and therefore reduced mass.

In some embodiments, the magnitude of the ion decay when entering an orbital electrostatic trap can be determined. The magnitude of the ion decay can be relative to the pressure in the ion store and/or mass spectrometer. In some embodiments, by repeating an analysis among a plurality of trapping gas pressures, and identifying a “zero time” intercept for each setting, e.g., a time and/or pressure at which the ion does not have kinetic energy losses. In some embodiments, ions may have more energy when entering an orbital electrostatic analyzer at lower pressures, which may result in higher apparent charges and mass. In some embodiments, ions may have reduced energy when entering an orbital electrostatic analyzer at higher pressures, which may result in lower apparent charges and mass. Without being bound by theory, by determining the effect of pressure on the absolute mass spectrum peak position the effect of the ion decay when entering an orbital electrostatic trap can be accounted for due to the capability of extrapolating to the “zero time”. Moreover, and without being bound by theory, extrapolating the “zero time” may allow for calibration to account for collisional kinetic energy losses that ions undergo when entering an orbital electrostatic trap and/or when in the orbital electrostatic trap.

In another embodiment, an ion decay event may occur when disposed in an orbital electrostatic trap. The magnitude of the ion decay when in the orbital electrostatic trap can be relative to the time the ion resides within the orbital electrostatic trap. As the transient acquisition progresses, the ion can experience more collisions in the orbital electrostatic trap. In some embodiments, the magnitude of the ion decay event when disposed in the orbital electrostatic trap can be determined by looking at how STORI slopes and corresponding masses of many ions evolve over time as the transients progress. For example, STORI plots of many ions can be segmented into multiple time windows, and the resulting absolute mass spectra from each time window can be compared. In some embodiments, a gradual loss in kinetic energy from collisions can result in a lower apparent mass as time progresses, in which by extrapolating back to “zero time”, the mass may be determined prior to kinetic energy losses in the orbital electrostatic analyzer, e.g., without collisional effects.

In some embodiments, the operation of charge determination module 220 may determine the STORI plot and apply one or more corrections and/or calibrations prior to determining the charge state. The one or more corrections and/or calibrations may correct the mass value to account for decays such that the undecayed ion, e.g., ion prior to ion decay events, may be properly determined. The charge and/or mass value may then be determined based on the corrected charge state value after adjusting for the kinetic energy losses occurring in the ion store, e.g., ion store kinetic energy loss, and/or kinetic energy losses occurring in the orbital electrostatic analyzer.

In another application of the STORI plot technique, the distribution of ion lifetimes of an ion of interest can be determined by repeated transient acquisitions and examination of the resultant STORI plots to identify when the decay event occurred, as evidenced by a change in plot slope. In some embodiments, the primary cause of ion decay events is collisions with background neutrals, in which the collision cross section can be determined based on the lifetime distribution and/or the transient acquisition time of the ion of interest in the orbital electrostatic analyzer. Without being bound by theory, the collision cross section may be determined in a fashion similar to ion mobility spectrometry.

### EXAMPLES

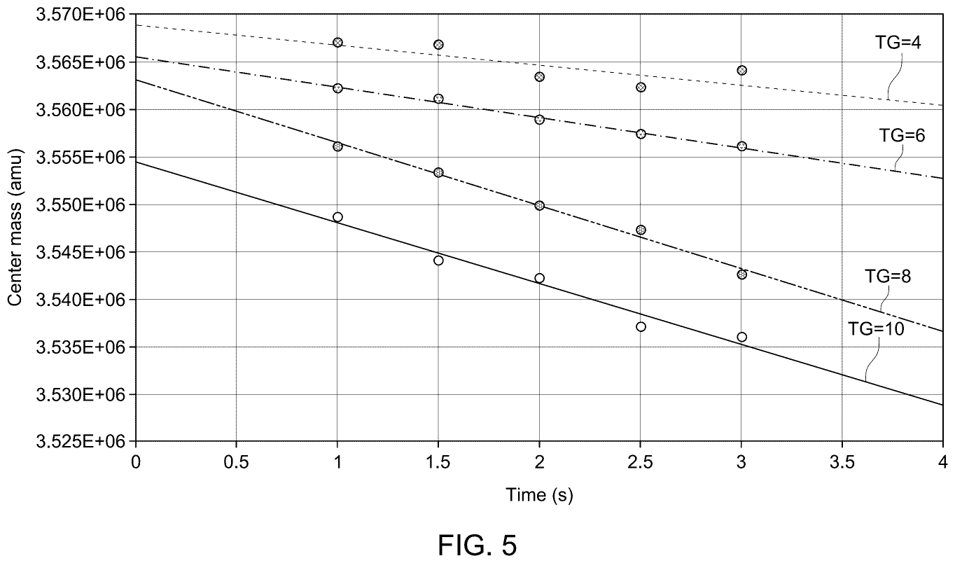

Now referring to FIG. 5, an exemplary CDMS signal variation as a result of ion decay is shown. Empty adeno-associated virus (AAV) capsid samples were analyzed via CDMS on an orbital electrostatic analyzer. Four-second-long transients were acquired under conditions that provided single ions in any m/z bin. The absolute mass spectra of 0.5 second time windows were fit to determine a mass shift over the course of the transient. The process was repeated at various trapping gas setpoints, from 4.0 to 10.0 arbitrary units. Without being bound by theory, at higher trapping gas settings, e.g., 8 and/or 10, more gas is residing in the orbital electrostatic analyzer, thereby causing faster kinetic energy losses compared to lower trapping gas settings.

Furthermore, and still referring to FIG. 5, the intercept, indicating the “zero time” of each curve decreased with higher trapping gas pressure. Without being bound by theory, the ions experienced more collisions during the flight between the ion store and the orbital electrostatic trap, and therefore entered the orbital electrostatic trap with less kinetic energy. Each of these effects, e.g., faster kinetic energy losses and reduced “zero time” were corrected for to increase the accuracy and/or precision of ion charge and/or mass.

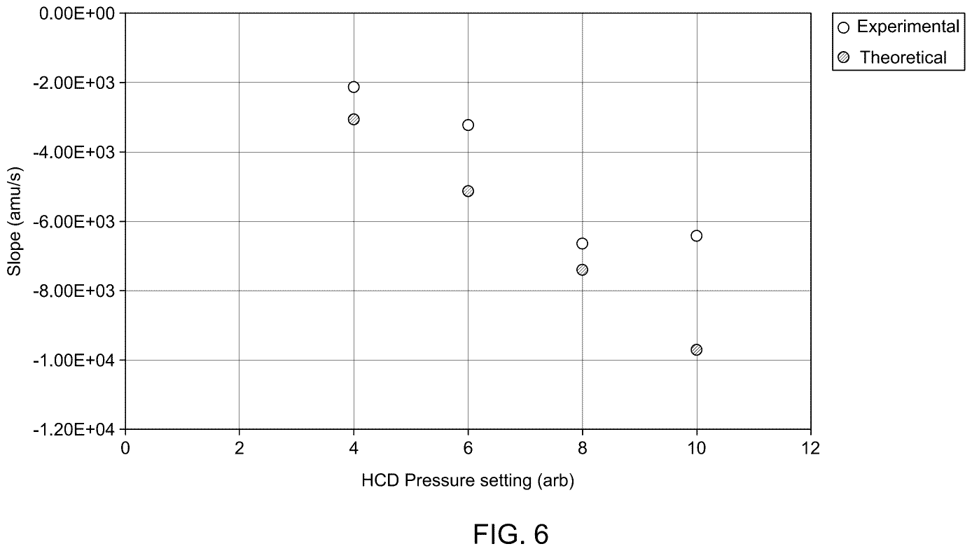

As transient time progressed, the estimated mass of the capsid samples decreased, e.g., a downward slope of each curve was determined, as shown in FIG. 6. The downward slope of each curve had a greater slope magnitude than a theoretical reference based on simulations. Without being bound by theory, the downward slope of each curve was due to kinetic energy losses inside the orbital electrostatic trap as well as kinetic energy losses that occurred in the ion store.

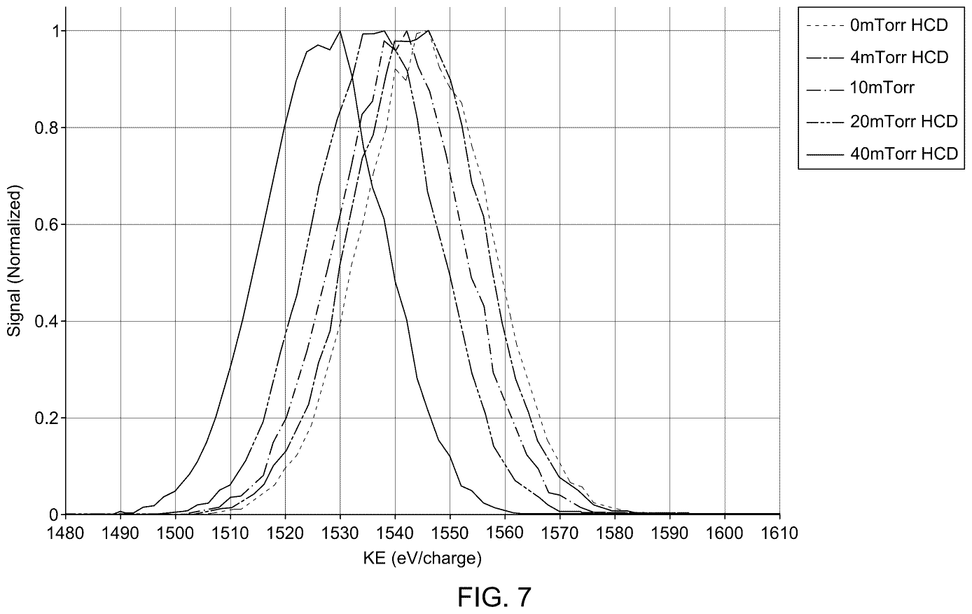

Now referring to FIG. 7, a simulation was performed to determine an amount of kinetic energy loss at varying pressures within an ion store to determine a kinetic energy loss that occurred in the ion store. The ion store pulse was set to 1550 volts (V), the push/pull voltage was set to 400 V, the mass of the ion traveling in the mass spectrometer was set to 3.6×10⁶ Daltons (Da), the charge was set to +150, the collision cross section (CCS) was set to 49,087 Å² the position of the ions was set to the center of the C-trap, and the distribution was 0.1 mm standard deviation in x, y, and z directions. Pressure in the ion store was set to 10 mTorr and pressure outside of the ion store was set to 0.2 mTorr. Higher collision dissociation (HCD) gas was introduced to an HCD cell at pressures of 0 mTorr, 4 mTorr, 10 mTorr, 20 mTorr, and 40 mTorr.

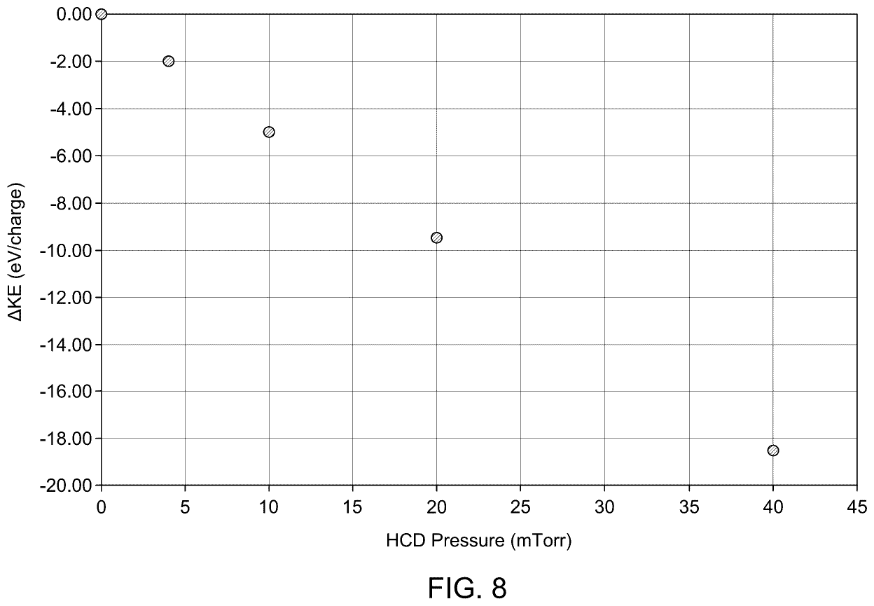

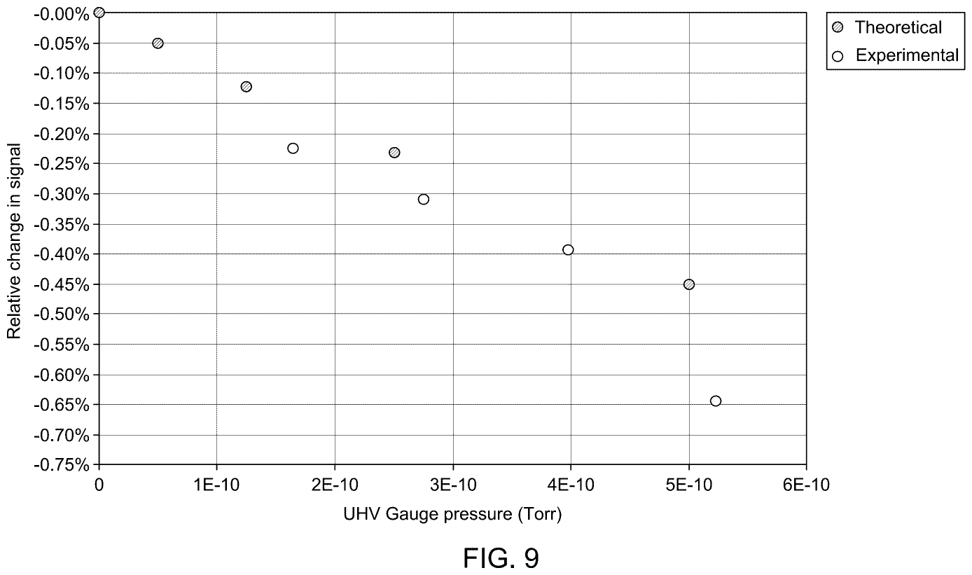

Ions were determined to have about 4.8 keV/charge when entering the orbital electrostatic analyzer at HCD gas pressures of 0 mTorr. At HCD gas pressures of 40 mTorr the kinetic energy to charge (eV/charge) was reduced by about 0.4% compared to when no HCD gas was introduced into the HCD cell, as shown in FIG. 8. Now referring to FIG. 9, the theoretical results of FIG. 8 aligned with the experimental results as shown in FIG. 6, thereby indicating an agreement between the theoretical and experimental values.

While the invention has been described above and depicted in the drawings in connection with its implementation in an orbital electrostatic trap having a quadro-logarithmic trapping potential, it should be understood that this implementation is described by way of an illustrative rather than a limiting example. The present disclosure may be implemented in any electrostatic trap or equivalent structure in which the confined ions undergo harmonic motion along a longitudinal axis, including traps in which the ions do not undergo orbital motion. An example of a non-orbital electrostatic trap that may be suitable for implementation of the present invention is the Cassinian trap described in Köster, “The Concept of Electrostatic Non-Orbital Harmonic Ion Trapping”, International Journal of Mass Spectrometry, V. 287, pp. 114-118 (2009), which is incorporated herein by reference.

In another embodiment, the present disclosure may be implemented in an electrostatic linear trap. The trapped ions may be trapped within an electrostatic linear ion trap, which may allow for the capture of all of the image current, and reduced capacitance compared to orbital electrostatic traps. Without being bound by theory, the use of an electrostatic linear ion trap can improve detection limits and improve precision.

One of ordinary skill in the art will recognize that due to small field faults arising from (for example) electrode machining tolerances, component misalignment, electrical noise and electrode truncation, the ions' motion along the longitudinal axis of the electrostatic trap or equivalent structure may exhibit slight deviation from purely harmonic (e.g., single-frequency sinusoidal) motion. However, such slight departures from pure harmonicity, which will occur in any real-world device, will not substantially reduce the performance of the methods outlined above for derivation of an ion's m/z and charge state. Thus, the term “harmonic”, as recited in the following claims, should be construed to encompass cases where small, operationally insubstantial departures from pure harmonic motion exist.

The discussion included in this application is intended to serve as a basic description. The present invention is not intended to be limited in scope by the specific embodiments described herein, which are intended as single illustrations of individual aspects of the invention. Functionally equivalent methods and components are within the scope of the invention. Various other modifications of the invention, in addition to those shown and described herein will become apparent to those skilled in the art from the foregoing description and accompanying drawings.

Not all of the various illustrated technical features and components are depicted and described for all possible embodiments. Features or components described for fewer than all of the illustrated embodiments are considered to be applicable to other embodiments, provided that they are not incompatible with those other embodiments.

Overall, the present disclosure provides methods to identify pressure influences on the absolute mass peak position by extrapolating a “zero time”, thereby accounting for kinetic energy losses in CDMS. The methods can calibrate and/or adjust the absolute mass peak position based on the collisional kinetic energy losses of an ion in charge detection mass spectrometry, e.g., collisional kinetic energy losses of an ion in an orbital electrostatic trap mass analyzer system. Additionally, the present disclosure can determine accurate charge assignments due to the calibration, thereby further clarifying the mass assignment of the ion in an orbital electrostatic trap mass analyzer system. Advantageously, the present disclosure can improve charge precision in CDMS processes and can determine a collision cross section for an ion and/or analyte during CDMS processes.

Any patents, patent applications, patent application publications or other literature mentioned herein are hereby incorporated by reference herein in their respective entirety as if fully set forth herein, except that, in the event of any conflict between the incorporated reference and the present specification, the language of the present specification will control.

What is claimed is:

1. A method for determining a kinetic energy loss, comprising:
injecting an ion population including an ion of interest into a trapping region and establishing an electrostatic trapping field within the trapping region;
generating a time-varying signal representative of a current induced on a detector based on the ion population;
generating a Selective Temporal Overview of Resonant Ion (STORI) plot based on the time-varying signal; and
identifying an ion decay event comprising a kinetic energy loss occurring in the trapping region.

2. The method of claim 1, further comprising determining a charge of the ion of interest, wherein determining the charge of the ion of interest comprises:
processing the time-varying signal to derive a frequency of the induced current; and
determining the charge of the ion of interest based on the STORI plot slope and the time-varying signal.

3. The method of claim 1, wherein identifying the kinetic energy loss further comprises determining a collision cross section of the ion of interest.

4. The method of claim 1, wherein identifying the kinetic energy loss comprises determining a pressure in the trapping region and determining the kinetic energy loss based on the pressure in the trapping region.

5. The method of claim 4, wherein the pressure in the trapping region is based on a trapping gas.

6. The method of claim 1, wherein identifying the kinetic energy loss comprises determining an acquisition time in the trapping region and determining the kinetic energy loss based on the acquisition time in the trapping region.

7. The method of claim 1, wherein the ion population is confined in an ion store prior to injection into the trapping region.

8. The method of claim 7, further comprising determining an ion store kinetic energy loss in the ion store prior to injection in the trapping region.

9. The method of claim 8, wherein identifying the ion store kinetic energy loss comprises determining a pressure in the ion store and determining the ion store kinetic energy loss based on the pressure in the ion store.

10. The method of claim 1, wherein the electrostatic trapping field is established in an annular region between an inner electrode and an outer electrode surrounding the inner electrode, and wherein the electrostatic trapping field has a potential distribution U(r,z), wherein U(r,z) is defined by:
$$U ( r , z ) = \frac{k}{2} ( {z}^{2} - \frac{{r}^{2}}{2} ) + \frac{k}{2} * ( {R}_{m} ) * ln ( \frac{r}{{R}_{m}} ) + C$$
where r is the position of the ion of interest along a radial axis, z is the position of the ion of interest along a central axis, k is a field curvature, C is a constant, and Rₘ is a characteristic field radius.

11. The method of claim 1, wherein the ion of interest comprises a protein, a protein complex, or a viral capsid.

12. A method for determining a kinetic energy loss, comprising:
storing an ion population including an ion of interest in an ion store;
injecting the ion population into a trapping region and establishing an electrostatic trapping field within the trapping region;
generating a time-varying signal representative of a current induced on a detector based on the ion population;
generating a generate a Selective Temporal Overview of Resonant Ion (STORI) plot based on the time-varying signal; and
identifying an ion decay event comprising a kinetic energy loss.

13. The method of claim 12, wherein identifying the kinetic energy loss further comprises determining a collision cross section of the ion of interest.

14. The method of claim 12, wherein identifying the kinetic energy loss comprises determining a pressure in the ion store and determining the kinetic energy loss based on the pressure in the ion store.

15. The method of claim 12, wherein the kinetic energy loss occurs in the ion store prior to injection in the trapping region.

16. The method of claim 12, wherein the kinetic energy loss occurs in the trapping region after injection of the ion of interest.

17. The method of claim 12, wherein the kinetic energy loss occurs between the ion store and the electrostatic trapping field during injection of the ion of interest.

18. The method of claim 12, further comprising determining a charge of the ion, wherein determining the charge of the ion comprises:
processing the time-varying signal to derive a frequency of the induced current; and
determining the charge of the ion based on the STORI plot slope and the time-varying signal.

19. The method of claim 12, wherein the ion of interest comprises a protein, a protein complex, or a viral capsid.

20. A method for determining a collision cross section of an ion of interest, comprising:
injecting an ion population including the ion of interest into a trapping region and establishing an electrostatic trapping field within the trapping region;
generating a time-varying signal representative of a current induced on a detector based on the ion population;
generating a generate a Selective Temporal Overview of Resonant Ion (STORI) plot based on the time-varying signal;
determining a collision cross section of the ion of interest.
